(19) United States
(12) Patent Application Publication
(10) Pub. No.: US 2005/0239720 A1
(43) Pub. Date: Oct. 27, 2005

(54) Combination of an agent that attenuates topoisomerase 1 activity and an agent that inhibits heat shock protein 90 for use in chemotherapy

(75) Inventor: John Jenkins, Liverpool (GB)

Correspondence Address: PILLSBURY WINTHROP SHAW PITTMAN LLP, 725 S. FIGUEROA STREET, SUITE 2800, LOS ANGELES, CA, 90017, US

(73) Assignee: UNIVERSITY OF LIVERPOOL Senate House, Liverpool (GB)

(21) Appl. No.: 10/511,001

(22) PCT Filed: Apr. 15, 2003
(86) PCT No.: PCT/GB03/01613

§ 371 Date: Jun. 13, 2005

(30) Foreign Application Priority Data
Apr. 15, 2002 (GB) 0208516.5

(51) Int. Cl. A61K 31/7048; A61K 31/4745

(52) U.S. Cl. 514/27; 514/283

(57) ABSTRACT

The present invention relates to the use of a first agent that attenuates Topoisomerase I (Topo I) activity and a second agent that inhibits Heat Shock Protein 90 (HSP90) for use in chemotherapy. The agents are particularly useful in the treatment of cancer and destruction of micro-organisms. The invention also relates to screening methods, diagnostic methods and methods for evaluating or monitoring chemotherapy regimens.

The present invention relates to the treatment of medical conditions using a combination of chemotherapeutic agents.

In general, when chemotherapy is used for the treatment of human cancers and the like, a combination of agents is employed. In the past, the reasoning behind the choice of which particular combinations of agents are used has been essentially a pragmatic decision, often based more on tolerances to toxicity rather than specific targets.

Recent studies of the process of carcinogenesis, have revealed that many of the genetic lesions involved, cause errors in the cell division/death pathways. The molecular changes that result from such lesions initiate the cancer process. Due to this the molecules involved in such changes provide potentially highly specific targets for chemotherapy. Using the targets identified by this approach new therapeutic agents may be introduced into the clinic. However, to achieve optimal clinical benefit from these agents, they may too need to be used in combination with other anticancer drugs. Again the choice of which particular combinations of agents are used has been a decision based more on tolerances to toxicity rather than specific targets.

There is also a need to develop new and improved antimicrobial agents. Antibiotic resistance is a growing problem and there is an increasing need to provide effective combination therapies.

According to a first aspect of the present invention, there is provided a use of a first agent that attenuates Topoisomerase I activity and a second agent that inhibits Heat Shock Protein 90 activity for the manufacture of a medicament for contemporaneous or sequential administration in chemotherapy.

According to a second aspect of the present invention, there is provided a method for conducting chemotherapy comprising contemporaneously or sequentially administering to a person or animal in need of said treatment a therapeutically effective amount of a first agent that attenuates Topoisomerase I activity and a second agent that inhibits Heat Shock Protein 90 activity.

According to a third aspect of the present invention, there is provided a composition for use in chemotherapy comprising therapeutically effective amounts of a first agent that attenuates Topoisomerase I activity and a second agent that inhibits Heat Shock Protein 90 activity and a pharmaceutically acceptable vehicle.

By “chemotherapy” we mean treatment of cells to cause a targeted cell death. Chemotherapy is required in cancer treatment where it is desirable to target transformed cells. Chemotherapy is also employed to treat infections caused by pathogens (e.g. bacterial, fungal or viral infections).

Topoisomerase I (Topo I) is an enzyme that catalyzes the transport of a single strand of DNA through another single strand of DNA. This works to alleviate the topological problems encountered by intracellular DNA. Topoisomerase I removes the negative (and positive, in eukaryotic topoisomerase I enzymes) supercoils from DNA. This is an extremely important reaction that allows RNA transcription and DNA replication to take place.

Topo I enzymes are monomeric and transiently break one strand of duplex DNA, allowing for single step changes in the linking number of circular DNAs (the number of times on strand of DNA crosses the other). Topo I can be divided into two subfamilies: Type IA and Type IB.

Type IA enzymes require magnesium and a single-stranded segment of DNA;

additionally they form a covalent intermediate with the 5′ end of the broken DNA strand and relax only negatively supercoiled DNA. Type IB topoisomerase I enzymes do not require any metal cofactors, work on double stranded DNA as well, form a covalent intermediate with the 3′ end of the broken strand, and are able to relax both positive and negative supercoils.

Topo I must also seal the break in the DNA. This reversible breakage is achieved because Topo I maintains the high energy status of the phosphodiester bond during unwinding. Topo I uses an enzyme residue (typically a tyrosine) to break DNA. A new enzyme-DNA phosphodiester bond is formed in the process. The covalent DNA-enzyme intermediate can be readily attacked by the free end of the DNA because the enzyme-DNA phosphodiester bond is of comparable energy to the orginal bond.

Sequences for Topoisomerase I are known to the art. Examples of sequences for known Topo I enzymes may be found in the following papers/gene databases:

(a) Human Topo I

D'Arpa et al. (1988) Proc Natl Acad Sci U S A 85(8):2543-7; NCBI pubmed nucleotide LOCUS HUMTOPI, ACCESSION J03250
   (b) Yeast Topo I
Thrash et al (1985) Proc Natl Acad Sci U S A 82(13):4374-8; NCBI pubmed nucleotide LOCUS YSCTOPI, ACCESSION K03077
   (c) E.coli Topo I
Tse-Dinh et al. (1986) J Mol Biol 191(3):321-31; NCBI pubmed nucleotide LOCUS ECTOPA, ACCESSION X04475 X12873

Human Topo I is of considerable biomedical importance because it is the main target of camptothecin (CPT) family of anticancer drugs. These drugs act by prolonging the lifetime of the nicked intermediate in the Topo I reaction which are presumed to form obstacles to the advancement of transcription and replication complexes that eventually lead to DNA damage and cell death.

Heat Shock Protein 90 (HSP90) consists of a highly conserved, 25 kDa N-terminal domain connected to a highly conserved, 55 kDa C-terminal region by a ‘charged linker’, which is variable in both length and composition among species and isoforms. The eukaryotic HSP90s are essential and ubiquitous molecular chaperones with key roles in the folding, activation and assembly of a range of client proteins typically involved in signal transduction, cell cycle control or transcriptional regulation.

Sequences for HSP90 are also known to the art. Examples of known HSP90 proteins may be found in the following papers/gene databases:
(a) Human HSP90 beta: Rebbe et al. Gene 1987;53(2-3):235-45; GENBANK/M16660; and NCBI PubMed nucleotide LOCUS HUMHSP90
(B) A bacterial homologue of HSP90 from E.coli (HtpG): Nemoto et al. Eur J Biochem. 2001 October;268(20):5258-69; swissprot: locus HTPG_ECOLI, accession P10413 (protein accession number); and NCBI PubMed protein LOCUS HTPG_ECOLI

Heat Shock proteins exert their effect under conditions of stress such as heat shock, oxidative, chemical and other stress situations. The biochemical function of HSP90 is catalysing the correct folding and maturation of a number of protein substrates. Without the function of HSP90 the abnormal conformation of the partner proteins would target them for proteolytic degradation.

HSP90 is known to bind to mediators of signalling pathways and other proteins but it is not known to the art that HSP90 may interact with Topo 1. However the inventors have established that HSP90 and Topo I interact.

The inventor has found that the combined use of a first agent that attenuates Topoisomerase I activity and a second agent that inhibits Heat Shock Protein 90 activity is highly effective for effecting chemotherapy. The first and second agents may be administered contemporaneously (e.g. as a composition according to the third aspect of the invention) or sequentially. If administered sequentially the first and second agents should be therapeutically active within the subject being treated at the same time.

Chemotherapy with first and second agents according to the invention is particularly useful because such therapy results in surprising synergistic actions. The inventors have found that disruption of the interaction between Topo I and HSP90 causes an increase in DNA damage and thereby kills 3-5 time (or more) the number of proliferating cells in comparison with what is achievable with a monotherapy. Furthermore satisfactory therapy may be effected using lower doses than would be required in a monotherapy. This has the advantage that the toxic side-effects associated with high doses of chemotherapeutic agents may be obviated or reduced.

For instance, damage to health tissues (and other associated side effects of high dose chemotherapy—e.g. sickness, hair loss) may be reduced in human cancer chemotherapy by using lower doses of the combined agents according to the invention (than would be required in a monotherapy) without comprising the efficacy of the treatment.

The invention is based upon studies that have been orientated towards the rational design of chemotherapeutic regimens. The inventor realised that drug development up to the present time has only been directed against single molecule targets and that rational selection of combination chemotherapy may be based on investigating the mechanisms of action of chemotherapeutic agents and identifying potential interaction at the cellular targets of such agents. The inventor's studies established that Topo I and HSP90 interact and lead to the realisation that a combination of agents that specifically inhibit the individual proteins will have great efficacy in chemotherapy. Further experimentation (see the Example) established that treatment of cells with a combination of agents according to the invention was highly effective as a chemotherapy. Furthermore the combination surprisingly represented a synergistic effect. Although the inventor does not wish to be bound by any hypothesis, it is believed that disrupting the interaction between the Topo I and HSP90 allows the generation of more DNA damage, thus killing the dividing cells, than would be possible using the agents in monotherapy. We believe the agents have such efficacy because two targets in a single pathway (the stress response pathway) are modulated.

Three papers in the prior art contemplate the use of HSP90 inhibitors in combination with other chemotherapeutic agents.

(a) Munster et al. (Clin Cancer Res 2001 August;7(8):2228-36) discloses that ansamycin antibiotics such as 17-AAG (an HSP90 inhibitor) and Doxorubicin may be combined in chemotherapy.

(b) Blagosklonny et al. (Leukemia 2001 October;15(10):1537-43) discloses that the ansamycin antibiotic (geldanamycin—an Hsp90 inhibitor) sensitises cells to the effects of Taxol or doxrubicin.

(c) Neckers (Trends in Molecular Medcine 2002 Vol(8) s55-s61) discloses that HSP90 inhibitors could increase the efficacy of certain chemotherapeutic agents.

However none of the papers contemplate modulation of Topo I. Furthermore there is no suggestion in any of these papers that agents that modulate Topo I may be combined with HSP 90 inhibitors according to the present invention and be used in chemotherapy to result in the sort of surprising and synergistic results reported herein.

A skilled person may be motivated to try combination therapies of an HSP90 inhibitor with a wide variety of other chemotherapeutic agents. However most of such combinations would have no beneficial or synergistic effect and the inventor believes that the efficacy of the specific combination of first and second agents according to the present invention would have been surprising to a skilled person.

Several classes of compound may be used according to the invention as the first agent. These compounds include:
(i) compounds that bind to Topo I and inhibit its activity (e.g. competitive inhibitors; allosteric inhibitors, cleavable complex inhibitors etc);
(ii) compounds which prevent the transcription, translation or expression of Topo I (e.g. ribozymes or antisense DNA molecules e.g. antisense crossing the first intron/exon boundary);
(iii) compounds which inhibit release of Topo I from intracellular stores; and
(iv) compounds which increase the rate of degradation of Topo I.

Examples of compounds that may be used as first agents are well known to the art. For instance, Pommier et al. (Biochimica et Biophysica Acta (1998) 1400 p83-106) disclose drugs targeted to Topo I as well as mechanism of action for Topo I. Such drugs are incorporated herein by reference as examples of preferred first agents.

Preferred compounds may attenuate the activity of human Topo I.

The compound may be a Topo I poison or a Topo I suppressor—e.g. as disclosed in Table 1 Pommier et al. (supra).

A preferred first agent is Gemcitabine (2′,2′-difluoro-2′-deoxycytidine). Gemcitabine is an antimetabolite that poisons Topo I (see Pourquier et al. Clin Cancer Res 2002 August 8(8) p2499-2504)

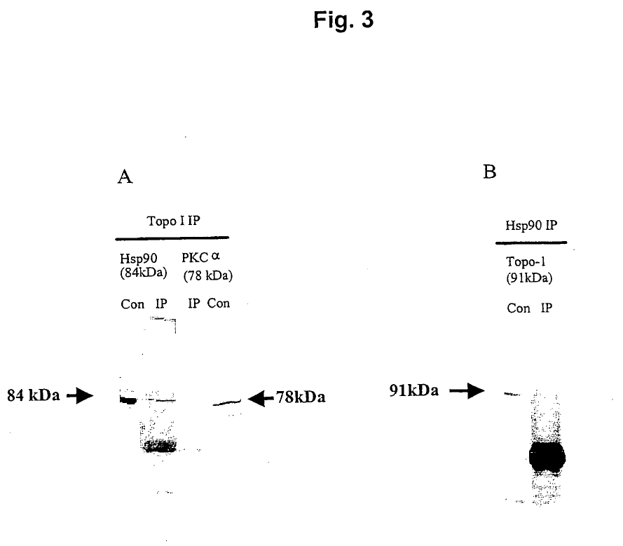

It is preferred that the first agent is Camptothecin (NSC 94600; and CAS Registry Number: 7689034) or a derivative thereof. Examples of preferred derivatives of Camptothecin are disclosed in Pommier et al. (supra) e.g. see FIG. 3 of the paper.
Camptothecin has the following names and structure:
Camptothecin
Camptothecine (8CI)
CAMPTOTHECIN
NSC 100880
1H-Pyrano[3′,4′:6,7]indolizino[1,2-b]quinoline-3,14(4H,12H)-dione, 4-ethyl-4-hydroxy-, (S)-(9CI)
20(S)-Camptothecine
21,22-Secocamptothecin-21-oic acid lactone

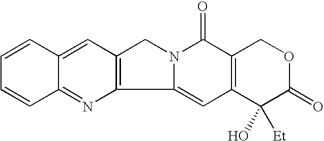

Another preferred first agent is Topotecan (NSC 609699) or a derivative thereof. Topotecan has the following names and structure:

Topotecan

9-Dimethylaminomethyl-10-hydroxycamptothecin, HCl salt

1H-Pyrano[3′,4′:6,7]indolizino[1,2-b]quinoline-3,14(4H,12H)-dione,

4-ethyl-4,9-dihydroxy-10-[(dimethylamino)methyl]-, HCl salt (S)

Hycamptamine

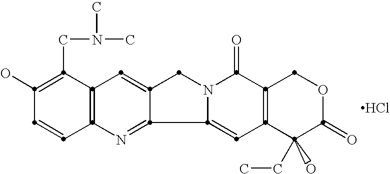

Another preferred first agent is Irinotecan or a derivative thereof. Irinotecan has the following names and structure:
Irinotecan

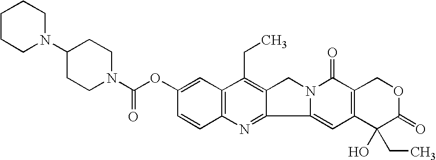

A further preferred first agent is Camptosar (CPT-11_or a derivative thereof.
Camptosar (CPT-11)

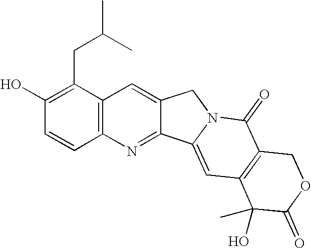

Several classes of compound may be used according to the invention as the second agent. These compounds include:
(i) compounds that bind to HSP90 and inhibit its activity (e.g. competitive inhibitors or allosteric inhibitors);
(ii) compounds which prevent the transcription, translation or expression of HSP90 (e.g. ribozymes or antisense DNA molecules);
(iii) compounds which inhibit release of HSP90 from intracellular stores; and
(iv) compounds which increase the rate of degradation of HSP90.

Geldanamycin and its derivatives (e.g. 17-Allylamino, 17-demethoxygeldanamycin-17-AAG or Macbecin II) are preferred second agents for use according to the present invention.

Macbecin II

Geldanamycin, 18,21-didehydro-6,17-didemethoxy-18,21-dideoxo-18,21-dihydroxy-15-methoxy-6-methyl-11-O-methyl-, (6S,15R)-(9CI)
Geldanamycin, 18,21-didehydro-6,17-didemethoxy-18,21-dideoxo-18,21-dihydroxy-15-methoxy-6-methyl-11-O-methyl-
Geldanamycin, 18,21-didehydro-6,17-didemethoxy-18,21-dideoxo-18,21-dihydroxy-15-methoxy-6-methyl-11-o-methyl-, (6S,1 5R)-
   Macbecin II 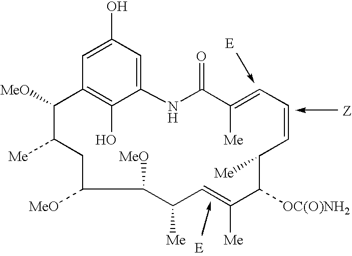
   2-Azabicyclo[16.3.1]docosane, geldanamycin deriv. (9CI)
   NSC 330500
   CAS Registry Number: 73341738 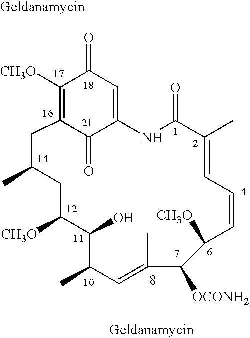
   NSC 122750

Radicicol may be used as a second agent according to the invention.

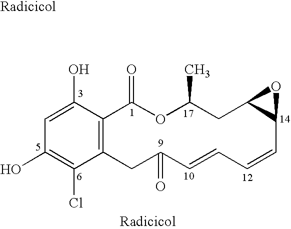

CNF-101 is a semi-synthetic derivative of Geldanamycin from Conforma Therapeutics (see www.conformacorp.com) and is a further preferred second agent.

It will be appreciated that agents may be developed that have a dual action in that they are able to attenuate Topo I activity and also inhibit Hsp90. Such agents may be used in an adaption of the present invention which involves the use of a single, dual action, agent only rather than separate first and second agents.

The first and second agents may be further combined with other therapeutics when there is a medical need. For instance, for certain medical conditions, the inventor has found even greater therapeutic efficacy when the agents are combined with a mediacment which suppresses apoptosis in non-cancerous tissue (eg pifithrin-α).

Agents which attenuate Topo I activity and inhibit Hsp90 may be used in chemotherapy to treat a number of conditions requiring the induction of targeted cell death. These include:

1) Cancer chemotherapy;

2) antibacterial treatments;

3) antifungal treatments;

4) the treatment of AIDS/HIV;

5) the treatment of multiple sclerosis; and

6) the killing and inhibition of proliferation of any organism.

When used to treat cancer, the agents are particularly effective for treating solid tumours such as bowel cancer, small cell and non-small cell lung cancer, head and neck cancer, breast cancer, bladder cancer and malignant melanoma.

The combined agents are also particularly effective for the treatment of paediatric tumours such as neuroblastoma and in the treatment of leukaemias and lymphomas, in which both proteins are contemporaneously or sequentially targeted.

The inventors have found that the combination of first and second agents improves the effectiveness of the agents for all known clinical applications for the agents.

When the agents are used to treat non-mammalian organisms, or to attack micro-organisms, it is preferred that the agents are effective for attenuating the activity of the species equivalent of Topo I or inhibiting the species equivalent of HSP90. For instance, when the agents are used as antibacterial agents it is preferred that they attenuate the activity of Topo 1A or an equivalent thereof (see Tse-Dinh et al. supra for E. coli TopA) and inhibit pHtpG (a bacterial equivalent of HSP 90).

We have found that the agents are particularly useful for arresting the growth or directly killing a number of bacteria. These include gram −ve and gram +ve bacterium.

The agents may be used to treat a number of bacterial infections in mammals (and particularly humans). Bacteria that may be attacked according to the invention are listed below. The conditions caused by such bacteria, and thereby treatable by the combination therapy according to the invention, are indicated in parentheses.

The following bacteria may be treated according to the invention:

Abiotrorphia (Reported infections—endophthalmitis, brain abscess, osteomyelitis);

Achromobacter (Reported infections—septicaemia, CAPD peritonitis, pneumonia, ear infection);

Acidaminococcus (Reported infections—abscesses, post surgical infections);

Acidovorax (Reported infections—wound infection, UTI, bacteraemia, meningitis, septic arthritis);

Acinetobacter (Reported infections—septicaemia, UTI, wound infections abscesses, endocarditis, meningitis, osteomyelitis);

Actinobacillus (Reported infections—periodontitis, endocarditis, abscesses, pericarditis, meningitis, septicaemia, pneumonia, empyema, hepatitis);

Actinobaculum (Reported infections—pyelonephritis);

Actinomadura (Reported infections—actinomycetoma, madura foot);

Actinomyces (Reported infections—actinomycosis);

Aerococcus (Reported infections—endocarditis, UTI, wound infection, meningitis, abscesses);

Aeromonas (Reported infections—wound infection, abscesses, septicaemia, acute diarrhoea, meningitis, leech bite infection, alligator bite infection, infections associated with aquatic exposure);

Afipia (Reported infections—cat scratch disease (A. felis), septic arthritis, bone marrow infection (A. broomeae), bone infection (A. clevelandensis);

Agrobacterium (Reported infections—endocarditis, CAPD peritonitis, UTI, line sepsis);

Alcaligenes (Reported infections—pneumonia, otitis, UTI, osteomyelitis, bacteraemia);

Alloiococcus (Reported infections—otitis media);

Amycolata (Please see Pseudonocardia);

Amycolatopsis (Species associated with infection—A. orientalis);

Anaerobospirillum (Reported infections—diarrhoea, bacteraemia);

Anaerorhabdus (Reported infections—lung abscess, appendix absecess, abdominal abscess);

“Anguillina” (Species associated with infection—“Anguillina coli”);

Arachnia (Species associated with infection—Arachnia propionica);

Arcanobacterium (Reported infections—septic arthritis (A. bernardiae and A. pyogenes), UTI and septicaemia (A. bernardiae), tonsillitis, cellulitis, lymphadenitis, brain abscess, septicaemia, osteomyelitis (A. haemolyticum);

Arcobacter (Reported infections—enteric infection (diarrhoea and abdominal cramps);

Arthrobacter (Reported infections—UTI, bacteraemia, Whipple's disease);

Atopobium (Reported infections—UTI, dental abscess, pelvic abscesses, wound infection);

Aureobacterium (With the exception of A. resistens, Aureobacterium spp. have been reclassified as members of the genus Microbacterium. The name A. resistens (which is vancomycin-resistent) was validly after other Aureobacterium spp. were reclassified as Microbacterium spp. Aureobacterium isolates have been misidentified as “Corynebacterium aquaticum”);

Bacillus (B. anthracis—the agent of anthrax B. thuringiensis, a biological insecticide has caused corneal infection);

Bacteroides (Reported infections—abscesses, bacteraemia, bite infections, wound infections, chronic otitis media, pelvic inflammatory disease);

Balneatrix (Reported infections—pneumonia, bacteraemia, meningitis);

Bartonella (Reported infections—Oroya fever and verruga peruana (B. bacilliformis), cat scratch disease (B. henselae), bacillary angiomatosis (B. henselae, B. quintana), trench fever (B. quintana), endocarditis (B. elizebethae), bacteraemia (B. vinsonii arupensis);

Bergeyella (Reported infections—wound infection, septicaemia, meningitis);

Bifidobacterium (Reported infections—bacteraemia, peritonitis, abscesses, otitis, paronychia);

Bilophila (Reported infections—appendicitis, abscesses, bacteraemia, biliary tract sepsis);

Branhamella (Species associated with infection—B. catarrhalis, this organism has been reclassified as Moraxella catarrhalis);

Borrelia (Species associated with relapsing fever, Reported infections—wound infection, septicaemia, meningitis);

Bordetella (Reported infections—respiratory tract infection (B. bronchiseptica, B. paralpertussis, B. pertussis), whooping cough (B. parapertussis, B. pertussis), bacteraemia, otitis, wound infection (B. hinzii, B. holmseii, B. trematum),

Brachyspira (Reported infections—intestinal spirochaetosis);

Brevibacillus (Reported infections—endophthalmitis, food poisoning, bacteraemia);

Brevibacterium (Reported infections—bacteraemia, meningitis, chest infection);

Brevundimonas (Reported infections—septicaemia);

Brucella (Reported infections—brucellosis);

Burkholderia (associated infections include lung infection, bacteraemia, endocarditis, septic arthritis, UTI, cystic fibrosis patients);

Buttiauxella (Reported infections—appendicitis, wound infection);

Butvrivibrio (Reported infections—endophthalmitis);

Calymmatobacterium (This taxon has been reclassified as Klebsiella granulomatis);

Campylobacter (associated with diarrhoea, bacteraemia, periodontitis, appendicitis, peritonitis and head and neck infections fever, meningoencephalitis, endocarditis, abscesses and abscess—zoonoses from mammals and birds,

Campylobacter butzleri was reclassified as Arcobacter butzleri C. cinaedi, C. fennelliae, C. pyloridis were reclassified as Helicobacter spp);

Capnocytophaga (wound infection, septicaemia, abscesses, meningitis, endocarditis—associated with dog bites systemic infections in neutropenic patients);

Cardiobacterium (Reported infections—endocarditis, meningitis);

Catonella (Reported infections—periodontitis);

Cedecea (Reported infections—bacteraemia);

Cellulomonas (reported cases of bacteraemia, meningitis cases of bacteraemia, endocarditis);

Centipeda (Reported infections—periodontitis);

Chlamydia (Reported infections—trachoma, genital infection, neonatal infection, lymphogranuloma venereum);

Chlamydophila (associated with abortion following contact with infected ruminants, associated with chest infection agent of psittacosis, a zoonosis from birds);

Chromobacterium (Reported infections—septicaemia, osteomyelitis, abscesses, eye infection);

Chyseobacterium (Reported infections—bacteraemia, meningitis, abdominal sepsis, wound infection, line infection);

Chryseomonas (Chryseomonas luteola has been reclassified as Pseudomonas luteola);

Citrobacter (Reported infections—UTI, meningitis, haemolytic-uraemic syndrome);

Clostridium (associated with wound infection, bacteraemia and abscesses, botulism, diarrhoea (usually antibiotic-associated) and pseudomembranous colitis, food poisoning, necrotising enterocolitis (pigbel, Darmbrand), gas gangrene—(C. histolyticum C. novyi, C. septicum, C. sordellii also associated with gas gangrene), tetanus);

Collinsella (Species associated with infection—Collinsella aerofaciens),

Comamonas (Reported infections—bacteraemia, conjunctivitis);

Corynebacterium (associated with infections such as septicaemia, peritonitis, eye infection, wound infection, endocarditis, osteomyelitis, septic arthritis, meningitis and abscesses diphtheria and cutaneous infection, tropical ulcer, septicaemia, pulmonary infection, lymphadenitis pharyngitis or diphtheria-like illness);

Coxiella (The agent of Q fever);

Cryotobacterium (Associated with periodontitis);

Delftia (Reported cases of bacteraemia and endocarditis);

Dermabacter (brain abscess, bacteraemia, wound infection);

Dermatophilus (Reported to cause cutaneous infection—zoonosis from cattle, sheep, goats and horses);

Desulfomonas (Associated with pilonidal abscess and peritonitis);

Desulfovibrio (Reported infections—bacteraemia, liver abscess);

Dialister (Reported infections—periodontitis);

Dichelobacter (Reported infections—pilonidal cyst, rectal fistula, wound infection);

Dolosicoccus (Reported infections—bacteraemia);

Dolosigranulum (Reported infections—spinal cord infection, eye infection);

Edwardsiella (Reported infections—wound infections, abscesses, gastroenteritis—associated with aquatic exposure and penetrating fish injury);

Eggerthella (Reported infections—rectal abscess);

Ehrlichia (Reported infections—Ehrlichiosis);

Eikenella (Reported infections—septicaemia, endocarditis, abscesses, septic arthritis);

Empedobacter (Species associated with infection—E. brevis);

Enterobacter (Associated infections—bacteraemia, respiratory tract infections, UTI—associated with nosocomial infection);

Enterococcus (Associated infections—bacteraemia, abscesses, endocarditis, meningitis, UTI, peritonitis, osteomyelitis, wound infection);

Erwinia (Associated infections—UTI);

Erysipelothrix (Associated infections—erysipeloid, septicaemia, endocarditis);

Escherichia (associated with UTI, bacteraemia, wound infection, meningitis, enteric infection, haemolytic uraemic syndrome);

Eubacterium (Associated infections—wound infection, abscesses, septicaemia, periodontitis);

Ewingella (Associated infections—septicaemia, wound infection, UTI);

Exiguobacterium (Species associated with infection—E. acetyliticum, E. aurantiacum),

Facklamia (Associated infections—UTI, bacteraemia, abscess);

Filifactor (Associated infections—gingivitis, periodontitis);

Flavobacterium (Associated infections—bacteraemia, diarrhoea);

“Flexispira” (Associated infections—bacteraemia, diarrhoea);

Francisella (associated with septicaemia and invaxsive systemic infection, tularaemia);

Fusobacterium (Associated infections—abscesses, bacteraemia, periodontitis, endocarditis, necrobacillosis);

Gardnerella (Associated infections—intrauterine and neonatal sepsis—associated with bacterial vaginosis);

Gemella (Associated infections—bacteraemia, endocarditis);

Globicatella (Associated infections—bacteraemai, UTI, meningitis);

Gordona (Associated infections—pulmonary infection, sternal wound sepsis, brain abscess, bacteraemia);

Haemophilus (associated with Brazilian purpuric fever; associated with sinusitis, otitis media, pneumonia, abscesses, endocarditis; the agent of chancroid; associated with bacteraemia, meningitis, epiglottitis, respiratory tract infection);

Hafnia (Associated infections—bacteraemia—has been associated with cases of diarrhoea);

Helicobacter (a zoonosis from dogs and hamsters cause of gastroenteritis; associated with septiciaemia and proctitis; sepicaemia in a neonate; gastritis);

Helococcus (associated with sebaceous cyst infection and breast absecess);

Holdemania (Species associated with infection—H. filiformis);

Ignavigranum (Associated infections—wound infection, ear abscess);

Johnsonella (Associated infections—perodontitis);

Kingella (Associated infections—septic arthritis, endocarditis);

Klebsiella (associated with UTI, bacteraemia, wound infection, respiratory tract infection; rhinoscleroma);

Kocuria (Species associated with infection—K. varians, K. kristinae);

Koserella (Associated infections—wound infection, septic arthritis);

Kurthia (bacteraemia and endocarditis; diarrhoea);

Kytococcus (Species associated with infection—K. sedentarius);

Lactobacillus (Associated infections—abscesses, bacteraemia, endometritis, endocarditis, lung infection, UTI—reported risk factors for infection, surgery, malignacy, diabetes mellitus, immunodeficiency);

Lactococcus (Associated infections—bacteraemia, endocarditis, UTI);

Lautropia (has been isolated from oral flora of an HIV patient and from sputum of a cystic fibrosis patient);

Leclercia (Associated infections—bacteraemia, wound infection);

Legionella (Associated infections—legionaires' disease, Pontiac fever);

Leminorella (Associated infections—UTI);

Leptospira (Associated infections—leptospirosis);

Leptotrichia (Associated infections—bacteraemia, endocarditis);

Leuconostoc (Associated infections—meningitis, bacteraemia, pulmonary infection);

Listeria (Associated infections—septicaemia, meningitis, intra-uterine infection, enteric infection);

Megasphaera (Associated infections—septicaemia, meningitis, intra-uterine infection, enteric infection);

Methylobacterium (Associated infections—bacteraemia, CAPD peritonitis);

Microbacterium (Associated infections—endophthalmitis, UTI, endocarditis, soft tissue infection, hypersensitivity pneumonitis, meningitis, CAPD peritonitis);

Micrococcus (Associated infections—bacteraemia, endocarditis, septic arthritis);

Mitsuokella (Species associated with infection—M. multiacida);

Mobiluncus (Associated infections—endometritis, chorioamnionitis—associated with bacterial vaginosis);

Moellerella (diarrhoea);

Moraxella (associated with—conjunctivitis, wound infection, endocarditis, abscesses, osteomyelitis);

Morganella (Associated infections—bacteraemia, UTI, wound infection);

Mycobacterium (Leprosy, cervical adenitis, Buruli ulcer, fish-tank granuloma, M. malmoense, M. szulgai M. kansasii, M. xenopi—associated with pulmonary infection, systemic infection in imunocompromised patients, post-inoculation infection);

Mycoolasma (Associated infections—respiratory infection, post-partum fever, pyelonephritis, pelvic inflammatory disease, myocarditis, pericarditis, meningitis);

Myroides (Associated infections—UTI, wound infection);

Neisseria (associated with meningitis, bacteraemia, endocarditis, osteomyelitis, agent of genital gonorrhoea, septicaemia, ophthalmia neonatorum, associated with septicaemia, meningitis, conjunctivitis, genital infection, epiglottitis)

Nocardia (nocardiosis);

Nocardiopsis (Associated infections—mycetoma, cutaneous infection, pulmonary infection, conjunctivitis);

Ochrobactrum (Associated infections—bacteraemia, endophthalmitis, liver abscess—reported association with nosocomial infections in debilitated patients);

Oeskovia (associated with meningitis, pyelonephrosis, CAPD peritonitis, endophthalmitis);

Oligella (associated with UTI, septicaemia—infection associated with urinary catheters);

Orientia (Associated infections—scrub typhus);

Paenibacillus (Associated infections—septicaemia, meningitis, pneumonia);

Pantoea (Associated infections—bacteraemia, endocarditis, wound infection, cellulitis, alligator bite infection, endophthalmitis);

Parachlamydia (Parachlamydia acanthamoebae has been associated with hypersensitivity pneumonitis (humidifier fever);

Pasteurella (Associated infections—wound infection, septicaemia, abscesses, pneumonia, endocarditis, meningitis—infections relate to spp);

Pediococcus (Associated infections—bacteraemia, abscesses, pulmonary infection—infections in debilitated patients);

Peptococcus (Peptococcus niger has been associated with anaerobic infections including intra-abdominal sepsis);

Photobacterium (Associated with necrotising wound infection);

Photorhabdus (Associated infections—bacteraemia, wound infection);

Plesiomonas (Associated infections—gastroenteritis, septicaemia, meningitis, endophthalmitis);

Porphyrimonas (Associated infections—mixed anaerobic infections at various sites, periodontitis, associated with bite infections (human and animal);

Prevotella (Associated infections—abscesses, bacteraemia, wound infection, bite infections, genital tract infections, periodontitis);

Propionibacterium (Associated infections—abscesses, endocarditis, bacteraemia, spetic arthritis, endophthalmitis, acne vulgaris);

Proteus (Associated infections—UTI, bacteraemia, wound infection, abscesses);

Providencia (Associated infections—UTI, wound infection, bacteraemia);

Pseudomonas (Reported infections—bacteraemia, UTI, wound infection, abscesses, septic arthritis, conjunctivitis, endocarditis, meningitis, CAPD peritonitis—nosocomial)

Pseudonocardia (Species associated with infection—P. autotrophica)

Pseudoramibacter (Associated infections—periodontal disease, wound infection, abscesses)

Psychrobacter (Associated infections—meningitis, bacteraemia, eye infection);

Rahnella (Associated infections—UTI, septicaemia);

Ralstonia (Associated infections—bacteraemia, UTI, meningitis, wound infection, peritonitis);

Rhodococcus (associated with bacteraemia, osteomyelitis, lung abscesses—infections of immunocompromised patients including AIDS);

Rickettsia (Associated infections—rickettsial spotted fever, tick typhus, tick bite fever, rickettsialpox);

Roseomonas (Associated infections—bacteraemia, wound infection, peritonitis);

Rothia (Associated infections—endocarditis, abscesses);

Ruminococcus (Associated infections—abdominal sepsis, abscesses);

Salmonella (Associated infections—gastroenteritis, enteric fever, osteomyelitis);

Selenomonas (Associated infections—bacteraemia, lung abscess—infections reported to be associated with malignancy or alcohol abuse);

Serratia (Associated infections—septicaemia, abscesses, bum infections, osteomyelitis);

Shewenella (associated with cases of intra-abdominal sepsis, meningitis and bacteraemia);

Shigella (Associated infections—enteric infection);

Simkania (Associated infections—bronchiolitis, pneumonia);

Slackia (Associated infections—periodontitis);

Sphingobacterium (Associated infections—bacteraemia, UTI, peritonitis);

Sphingomonas (Associated infections—septicaemia, UTI, wound infections, CAPD peritonitis—nosocomial infections);

Spirillum (Associated infections—rat bite fever);

Staphylococcus (Associated infections—Bacteraemia, wound infection, endocarditis, catheter-related sepsis,UTI, toxic shock syndrome, eye infection, osteomyelitis);

Stenotrophomonas (associated with various (mostly nosocomial) infections—bacteraemia, meningitis, wound infection, UTI and pneumonia);

Stomatococcus (Associated infections—endocarditis, meningitis, neutropenic sepsis);

Streptobacillus (Associated infections—rat bite fever, Haverhill fever);

Streptococcus (Associated infections—pharyngitis, bacteraemia, pyogenic infection, necrotising infection, septic arthritis, glomerulonephritis, meningitis, rheumatic fever, abscesses, endocarditis, pharyngitis, wound infection, pneumonia, pericarditis, CAPD, peritonitis, sinusitis, otitis, conjunctivitis);

Streptomyces (Associated infections—actinomycetoma);

Succinivibrio (Associated infections—bacteraemia);

Sutterella (Associated infections—appendicitis, peritonitis, abascesses, osteomyelitis);

Suttonella (Associated infections—endocarditis, eye infection);

Tatumella (Associated infections—bacteraemia, UTI);

Tissierella (Associated infections—bacteraemia);

Trabulsiella (Associated infections—diarrhoea);

Treponema (associated with periodontal disease, pinta, genital lesions, venereal and non-venereal endemic syphilis, yaws);

Tropheryma (associated with Whipple's disease);

Turicella (Associated infections—otitis, cervical abscess);

Ureaplasma (Associated infections—urethritis);

Vagococcus (Species associated with infection—V. fluvialis);

Veillonella (Associated infections—abscesses, bacteraemia);

Vibrio (The agent of cholera, associated with wound infection, bacteraemia, diarrhoea and septiciaemia, septicaemia, meningitis, endometritis);

Weeksella (associated with peritonitis and vaginal infections);

Xanthomonas (bacteraemia);

Yersinia (agent of plague, associated infections—enterocolitis, soft tissue infections, mesenteric lymphadenitis, enteric infection); and

Yokenella (Associated infections—bacteraemia, wound infection).

The combination therapy may also be used to treat fungal infections of a subject. The agents are effective against the following fungi: Candida spp, Aspergillus spp. Malassezia spp, Trichosporon spp, Fusarium spp, Paecilomyces spp and Acremonium spp, also Rhizopus, Mucor, Absidia, Blastomyces spp, Coccidiodes spp, Cryptococcus spp, Histoplasma spp

The inventor has also found that the combination therapy may also be used to treat a number of parasitic infections of a subject. For instance, the combination therapy is useful for treating malaria.

The inventors have found that the combination therapy is particularly useful for treating infection (e.g. local or systemic or deep systemic infections) associated with immune suppressed patients; urinary tract, bloodstream infections and pneumonia.

The agents may be used to treat existing medical conditions but may also be used when prophylactic treatment is considered medically necessary.

The agents used according to the invention may take a number of different forms depending, in particular on the manner in which they are to be used. Thus, for example, the agents may be in the form of a powder, tablet, capsule, liquid, ointment, cream, gel, hydrogel, aerosol, spray, micelle, liposome or any other suitable form that may be administered to a person or animal. It will be appreciated that the vehicle for the agents should be one which is well tolerated by the subject to whom it is given and enables delivery of the agent to the target tissue.

The agents may be used in a number of ways. For instance, systemic administration may be required in which case the agents may be contained within a composition, which may, for example, be ingested orally in the form of a tablet, capsule or liquid. Alternatively the agents may be administered by injection into the blood stream. Injections may be intravenous (bolus or infusion) or subcutaneous (bolus or infusion). The compounds may also be administered by inhalation (e.g. intranasally).

The agents may also be incorporated within a slow or delayed release device. Such devices may, for example, be inserted under the skin and the compound may be released over weeks or even months. The devices may be particularly advantageous when an agent is used which would normally require frequent administration (e.g. at least daily ingestion of a tablet or daily injection).

It is preferred that second agents according to the invention are initially dissolved in solvents such as DMSO before dilution in aqueous solution for the preparation of liquid medicaments.

The agents may be formulated as prodrugs. Such prodrugs may be stored as inactive and stable medicaments which are subsequently activated.

It will be appreciated that the amount of an agent required is determined by biological activity and bioavailability that in turn depends on the mode of administration and the physicochemical properties of the agents employed. The frequency of administration will also be influenced by the abovementioned factors and particularly the half-life of the agents within the subject being treated.

Known procedures, such as those conventionally employed by the pharmaceutical industry (e.g. in vivo experimentation, clinical trials etc), may be used to establish specific formulations of agents and precise therapeutic regimes (such as daily doses and the frequency of administration).

Generally, a daily dose of between 0.01 μg/kg of body weight and 1.0 g/kg of body weight of a first agent and a second agent may be used for chemotherapy depending upon which specific agents are used. More preferably the daily dose of each agent is between 0.1 μg/kg of body weight and 100 mg/kg of body weight.

Generally, a daily dose of 1 ng-1 g/M² (per agent) of both a first agent and a second agent may be used for chemotherapy in humans—depending upon which specific agents are used.

Purely by way of example suitable doses of first agents (e.g. Irinotecan, Topotecan, Camptothecin, Gemcitabine and derivatives and analogues thereof) according to the invention for treating a human cancer is 1 ng-1 g/M² IV (depending upon the health status of the individual) whereas 1 μg-1 g/kg is a suitable does for use in animals.

Purely by way of example suitable doses of second agents doses (for cancer chemotherapy or the treatment of micro organisms) according to the invention are:
(a) A suitable dose of Radicicol (or a derivative or analogue thereof) for treating a human cancer is 1 ng-1 gM² (depending upon the health status of the individual).
(b) A suitable dose of Geldanamycin for treating a human cancer is 1 ng-1 g/M².
(c) A suitable dose of 17-AAG for treating a human cancer is 1 ng-1 g/M².

For all agents it is preferred that about 1 μg-1 g/kg of a first or a second agent is used for vetinary purposes. For instance about 4-25 mg/kg of Geldanamycin may be used.

Daily doses may be given as a single administration (e.g. a daily tablet for oral consumption or as a single daily injection). Alternatively the agents used may require administration twice or more times during a day. A patient receiving treatment may take a first dose upon waking and then a second dose in the evening (if on a two dose regime) or at 3 or 4 hourly intervals thereafter. Alternatively a slow release device may be used to provide optimal doses to a patient without the need to administer repeated doses. A preferred route of administration is by intravenous infusion. Administration may be over several hours or even days.

A preferred means of using protein or peptide agents is to deliver such agents to the target tissue by means of gene therapy. For instance, gene therapy may be used to decrease expression of Topo I or HSP90, decrease expression of enzyme(s) responsible for the intracellular synthesis of Topo I or HSP90, increase expression of a protein which promotes breakdown of Topo I or HSP90. Therefore according to a fourth aspect of the present invention there is provided a delivery system for use in a gene therapy technique, said delivery system comprising:
(i) a first DNA molecule encoding for a protein which directly or indirectly attenuates Topoisomerase I activity; and
(ii) a second DNA molecule encoding for a protein which directly or indirectly inhibits Heat Shock Protein 90 activity;
   wherein said DNA molecules are capable of being transcribed to allow the expression of said proteins and thereby be effective for chemotherapy.

The delivery systems according to the fourth aspect of the invention are highly suitable for achieving sustained levels of a protein which are chemotherapeutically active over a longer period of time than is possible for most conventional therapeutic regimes. The delivery system may be used to induce continuous protein expression from cells in a target tissue that have been transformed with the DNA molecule. Therefore, even if the proteins have a very short half-life as agents in vivo, therapeutically effective amounts of the proteins may be continuously expressed from the treated tissue.

Furthermore, the delivery system of the invention may be used to provide the DNA molecules (and thereby the proteins which are active therapeutic agents) without the need to use conventional pharmaceutical vehicles such as those required in tablets, capsules or liquids.

The delivery system of the present invention is such that the DNA molecules are capable of being expressed (when the delivery system is administered to a patient) to produce proteins that directly or indirectly have activity for attenuating Topo I activity and inhibiting HSP90 activity. By “directly” we mean that the product of gene expression per se has the required activity. By “indirectly” we mean that the product of gene expression undergoes or mediates (e.g. as an enzyme) at least one further reaction to provide an agent effective for attenuating Topo I activity or inhibiting HSP90 activity.

The DNA molecules may be contained within a suitable vector to form a recombinant vector. The vector may for example be a plasmid, cosmid, virus or phage.

Such recombinant vectors are highly useful in the delivery systems of the invention for transforming cells with the DNA molecule.

The recombinant vector may also further comprise a promoter or regulator to control expression of the gene as required.

It will be appreciated that the first and second DNA molecules may be contained within a single vector and the expression thereof may be driven from either a single promoter or individual promoters. Alternatively the delivery system may comprise first and second DNA molecules contained within respective first and second expression vectors.

Recombinant vectors may also include other functional elements. For instance, recombinant vectors can be designed such that the vector will autonomously replicate in the cell. In this case, elements that induce DNA replication may be required in the recombinant vector. Alternatively the recombinant vector may be designed such that the vector and recombinant DNA molecule integrates into the genome of a cell. In this case DNA sequences which favour targeted integration (e.g. by homologous recombination) are desirable. Recombinant vectors may also have DNA coding for genes that may be used as selectable markers in the cloning process.

The DNA molecules may (but not necessarily) be one which becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells in the subject being treated. When this is the case, regulation of expression may be less important because expression of the DNA molecules will stop when the transformed cells die or stop expressing the proteins (ideally when chemotherapy is no longer required).

The delivery system may provide the DNA molecules to the subject without them being incorporated in a vector. For instance, the DNA molecules may be incorporated within liposomes or virus particles. Alternatively the “naked” DNA molecules may be inserted into a subject's cells by a suitable means e.g. direct endocytotic uptake.

The DNA molecules may be transferred to the cells of a subject to be treated by transfection, infection, microinjection, cell fusion, protoplast fusion or ballistic bombardment. For example, transfer may be by ballistic transfection with coated gold particles, liposomes containing the DNA molecules, viral vectors (e.g. adenovirus) and means of providing direct DNA uptake (e.g. endocytosis) by application of the DNA molecules directly to the target tissue topically or by injection.

The discovery that Topo I and HSP90 interact has enabled the inventor to develop a drug screening assay system for testing the efficacy of candidate drugs as chemotherapeutic agents. Therefore the two interacting proteins HSP90 and Topoisomerase I may be used as a complex target for new drug development in which both proteins are contemporaneously or sequentially targeted for new mammalian, fungal and anti bacterial agents.

According to a fifth aspect of the present invention there is provided a method of screening a first and a second compound, to test whether or not said compounds has efficacy for use in combination as a chemotherapy, comprising:
(i) exposing said compounds to Topoisomerase I and evaluating whether or not said compounds bind thereto;
(ii) exposing said compounds to Heat Shock Protein 90 and evaluating whether or not said compounds bind thereto; and
(iii) selecting a first and second compound, wherein at least one compound binds to Topoisomerase I and at least one compound binds to Heat Shock Protein 90 for use in combination as a chemotherapy.

It will be appreciated that the method according to the fifth aspect of the invention may be adapted such that it is used to test whether or not a single compound may have a novel use in chemotherapy. Therefore according to a sixth aspect of the invention there is provided a method of screening a compound, to test whether or not said compound has efficacy for use in chemotherapy, comprising exposing said compound to Topoisomerase I and Heat Shock Protein 90 to evaluate whether or not said compound prevents interaction between Topoisomerase I and Heatshock Protein 90.

Compounds screened according to the fifth or sixth aspects of the invention represent candidate chemotherapeutic agents. The screening methods are is based upon the inventors realisation that interaction between Topoisomerase I and Heat Shock Protein 90 is closely related to undesirable cell growth (carcinogenesis and the like). It will be appreciated that the pharmaceutical industry will be able to use the methods according to the fifth or sixth aspect of the invention to identify candidate medicaments for further investigation as anti-cancer agents.

A preferred technique for carrying out the methods of the fifth and sixth aspects of the invention is to exposure the compounds to be tested to Topo I and HSP90 used as binding partners in an interaction trap. Many forms of interaction trap are known to the art. Preferably a yeast two-hybrid interaction trap is employed. Yeast two-hybrid screening is a strategy for screening for interaction between proteins. Yeast two-hybrid screening used according to the invention may involve expression of translational fusions of (a) Topoisomerase I and part of a reporter gene; and (b) Heat Shock Protein 90 fused in-frame with the other part of the reporter gene. When the fusion proteins are expressed, interaction between (a) and (b) allows the reporter to assemble and generate a signal. Test compounds that represent candidate chemotherapeutic agents prevent interaction between (a) and (b) and may be identified because no reporter signal is produced from samples containing the candidate.

It will be appreciated that any other form of interaction trap may be used to put the invention into practice. Suitable examples included techniques such as mammalian two-hybrid, bacterial two-hybrid or alternatively various types of pull down assay.

When the methods relate to the disruption of protein-protein interactions based on the yeast two-hybrid technique it is preferred that yeast are used that are permeable to the tested compounds. Examples of drug permeable yeast which may be used according to the invention include MDS or ISE 2 mutations (e.g. strains carrying these mutations (ISE2), JJ700, BJ201). Suitable strains are disclosed in Hammonds et al. Antimicrob Agents Chemother. 1998 April;42(4):889-94.

It will be appreciated that the methods according to the fifth or sixth aspects of the invention may be adapted to identify compounds that promote interaction between Topoisomerase I and Heat Shock Protein 90 (rather than inhibit such interaction) Such an adapted test represents a good method for evaluating whether or not a test compound is likely to be carcinogenic. Therefore according to a seventh aspect of the present invention there is provided a method of screening a compound, to test whether or not said compound is carcinogenic, comprising exposing said compound to Topoisomerase I and Heat Shock Protein 90 to evaluate whether or not said compound promotes interaction between Topoisomerase I and Heat Shock Protein 90.

Accordingly any compound, identified according to the seventh aspect of the invention, that promotes interaction between Topoisomerase I and Heat Shock Protein 90 is likely to be carcinogenic. The method may be used to screen compounds to assess whether or not they are safe to be used by the public. For instance cosmetics, foodstuffs, candidate therapeutic agents etc may all be tested to investigate whether or not they may cause cancer. The method according to the seventh aspect of the invention may also be used for environmental monitoring. For instance, the test may be used to evaluate whether or not effluent from a factory may contain carcinogenic compounds.

The discovery that Topo I and HSP90 interact has further enabled the inventor to develop a test whereby the measurement of HSP90 and Topo I protein levels in cells is used as a diagnostic aid. According to an eighth aspect of the present invention there is provided an in vitro method for diagnosing whether or not a subject has, or is likely to develop cancer, comprising:
(i) detecting the level of activity or expression levels of HSP90 and Topoisomerase I from a sample of cells from said subject; and
(ii) comparing the level of activity or expression levels of HSP90 and Topoisomerase I in said sample relative to activity expression levels of HSP90 and Topoisomerase I from a non-cancerous sample.

The method according to the eighth aspect of the invention indicates that a subject is at risk of developing cancer if the activity or expression levels of Topo I or HSP90 are raised relative to control values (e.g samples from an individual without cancer or from non-cancerous tissues from the subject).

Preferably a first sample is taken from a tissue which is suspected to be cancerous and a second sample is taken from normal tissue (i.e. non-cancerous tissue) from the same subject.

The method according to the eighth aspect of the invention may be adapted for determining the sensitivity of a subject to a specific combination of first and second agents according to the invention (i.e. an HSP90 inhibitor and a Topo I inhibitor). Thus according to a ninth aspect of the present invention there is provided an in vitro method for evaluating the suitability of chemotherapeutic treatment for administration to a subject, comprising:
(i) detecting the level of activity or expression levels of HSP90 and Topo I from a sample of cells from said subject; and
(ii) comparing the level of activity or expression levels of HSP90 and Topo I in said sample relative to activity expression levels of HSP90 and Topo I from a non-cancerous sample.

According to a tenth aspect of the present invention there is provided an in vitro method for monitoring the effectiveness of a chemotherapy for treating a subject, comprising:
(i) detecting the level of activity or expression levels of HSP90 and Topo I from a sample of cells from said subject; and
(ii) comparing the level of activity or expression levels of HSP90 and Topo I in said sample relative to activity expression levels of HSP90 and Topo I from a non-cancerous sample.

The invention will be further illustrated with reference to non-limiting Examples and figures, in which:

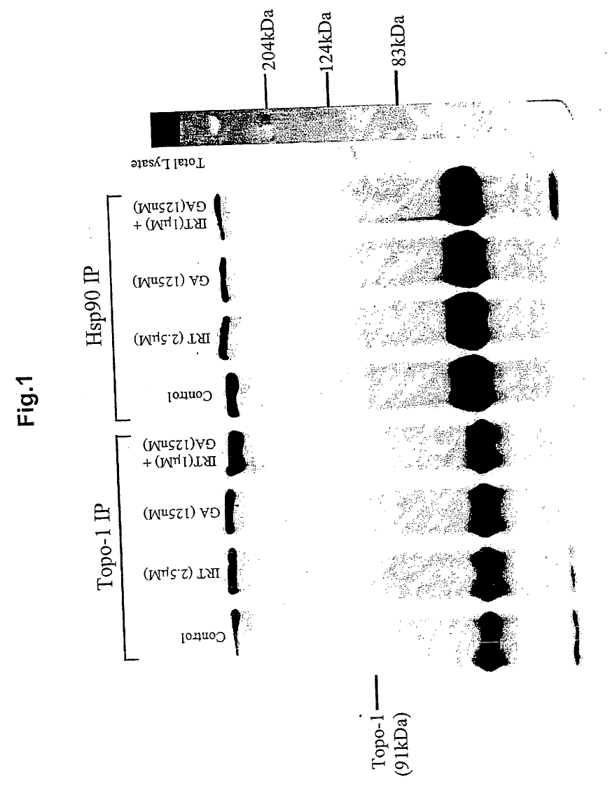

FIG. 1 illustrates the results of an immuno-precipitation assay with an HSP90 antibody in which protein from HCT116p53wt was subjected to a Western blot using Topo I as a probe as described in Example 1;

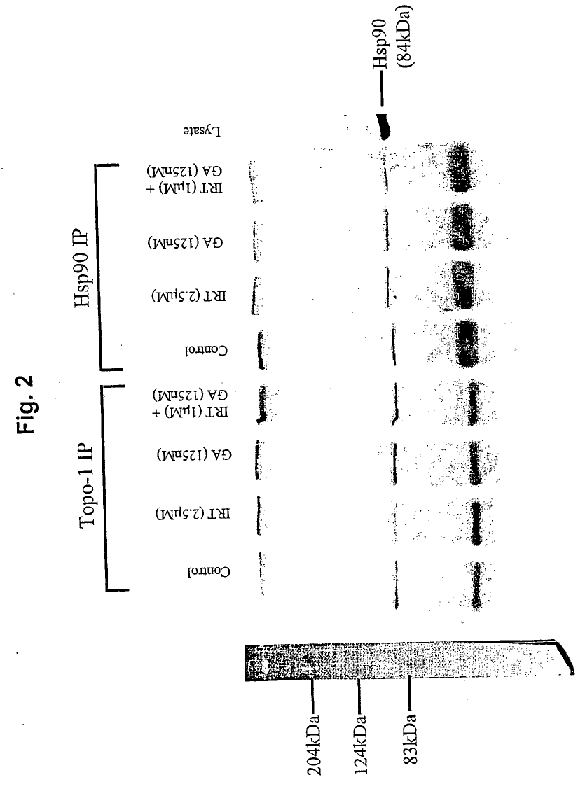

FIG. 2 illustrates the results of an immuno-precipitation assay with a Topo I antibody in which protein from HCT116p53wt was subjected to a Western blot using HSP90 as a probe as described in Example 1;

FIG. 3 illustrates the results of co-immunoprecipitation (IP) western blots from HCT116 extracts: (A) Immunoprecipitation with an anti-topoisomerase I antibody and probed with an antibody against Hsp90; as a negative control the blot was also probed with an antibody against PKCα; a non-reactive antibody was used as a non-specific binding control; and (B) an IP with anti Hsp90 antibody probed with a topoisomerase I antibody as described in Example 1;

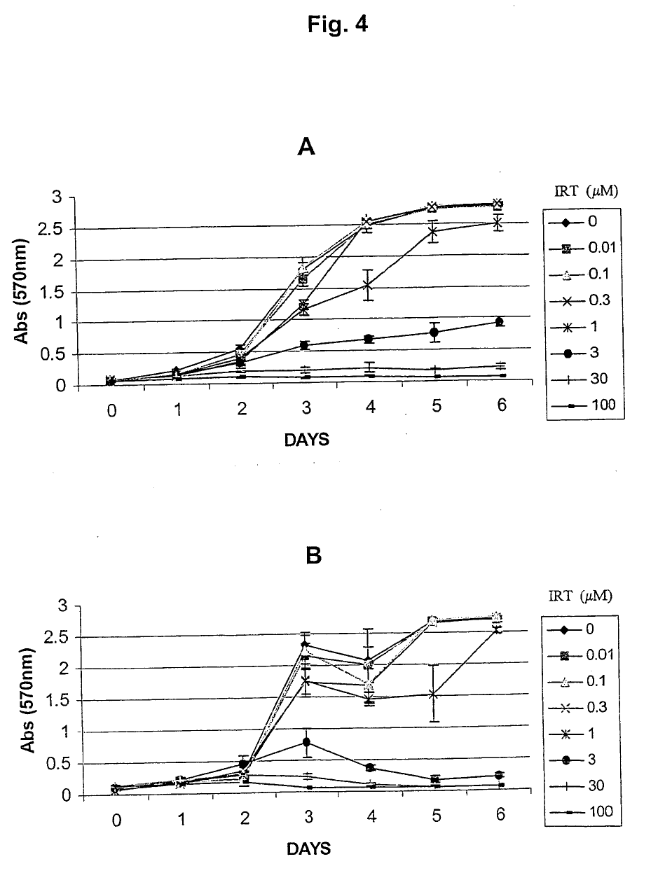

FIG. 4 illustrates the effect of Irinotecan alone on cell growth in (A) HCT116 p53 Wild type (WT) cells and (B) HCT116 p53 knockout cells as described in Example 2;

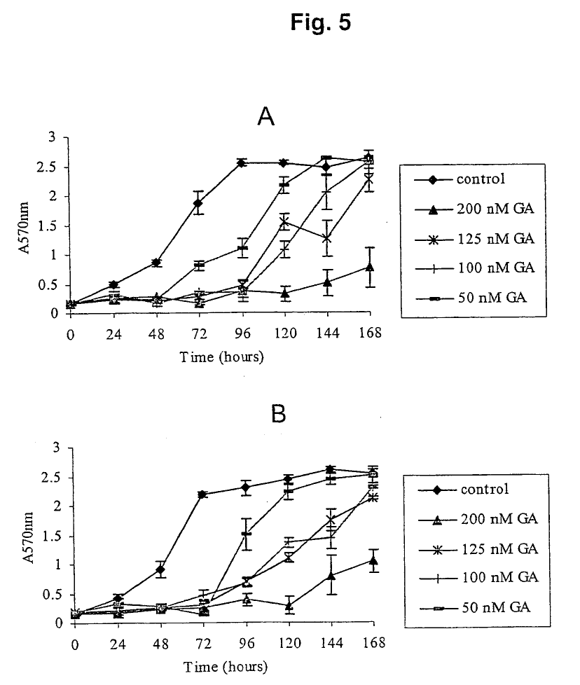

FIG. 5 illustrates the effect of Geldanamycin alone on cell growth in (A) HCT116 p53 Wild type (WT) cells and (B) HCT116 p53 knockout cells as described in Example 2;

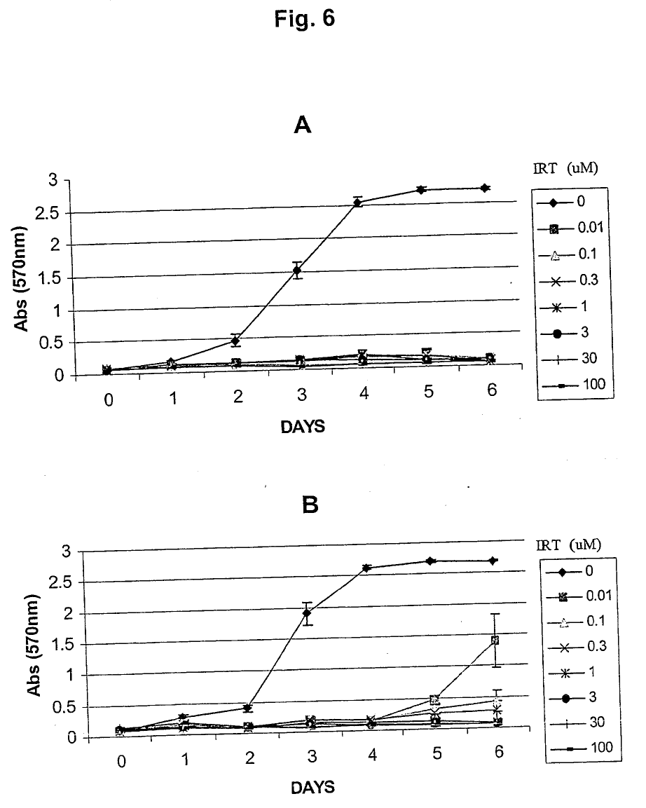

FIG. 6 illustrates the effect of a combined treatment of Irinotecan and Geldanamycin on cell growth (the effect being independent of p53 status) in (A) HCT116 p53 Wild type (WT) cells and (B) HCT116 p53 knockout cells as described in Example 2;

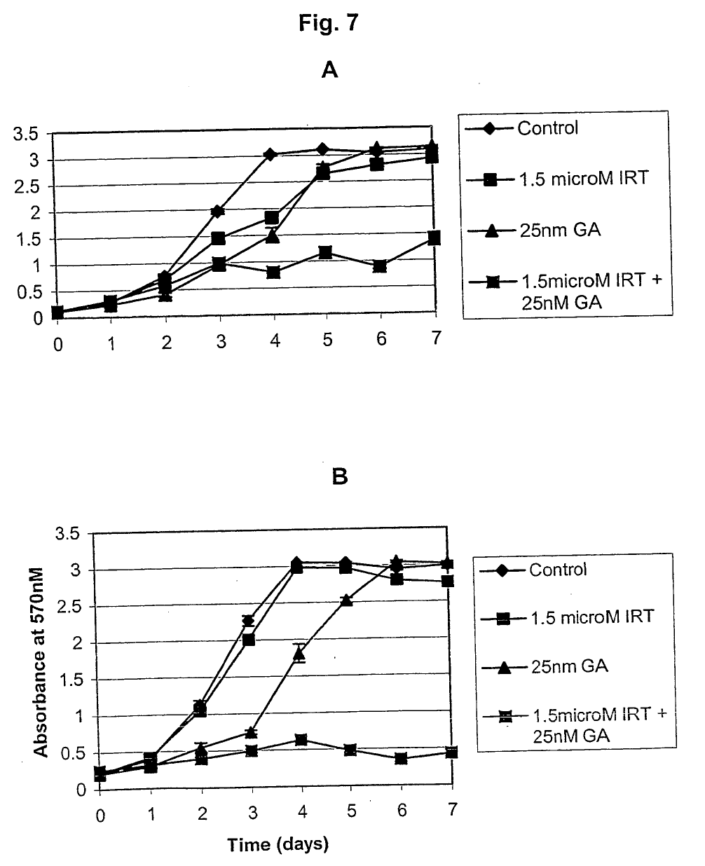

FIG. 7 illustrates the growth inhibition effect of 1.5 μM Irinotecan+25 nM Geldanamycin Combination Treatment on (A) HCT116 p53 WT cells and (B) p53 KO cells as described in Example 2;

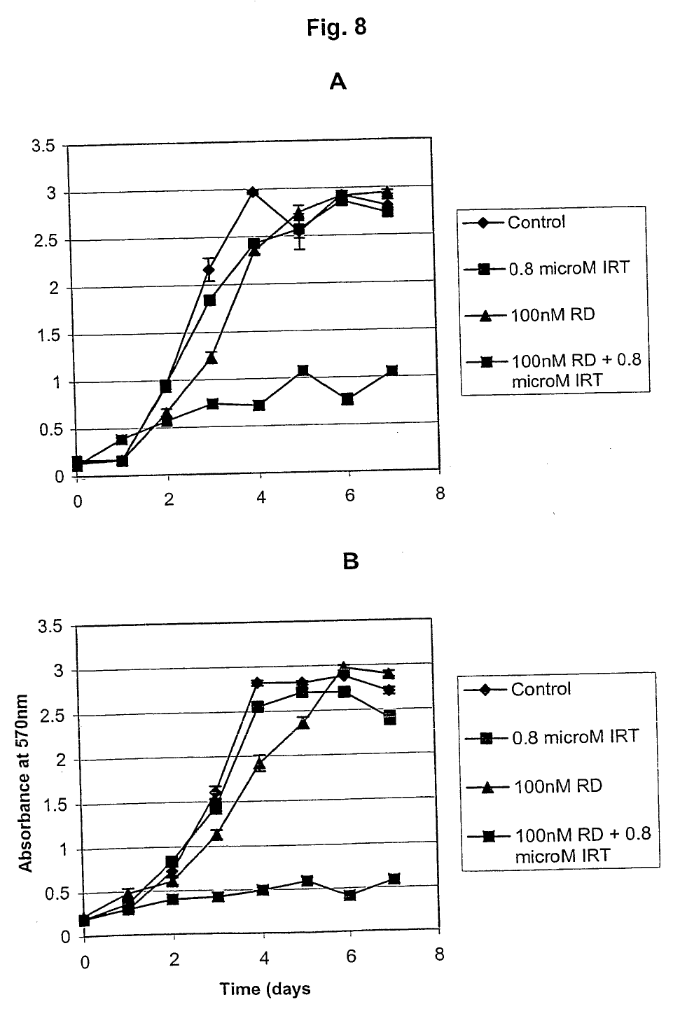

FIG. 8 illustrates the inhibitory effect of 0.8 μM IRT and 100 nm RD Combination Treatment on (A) HCT116 p53 WT cells and (B) p53 KO cells as described in Example 2;

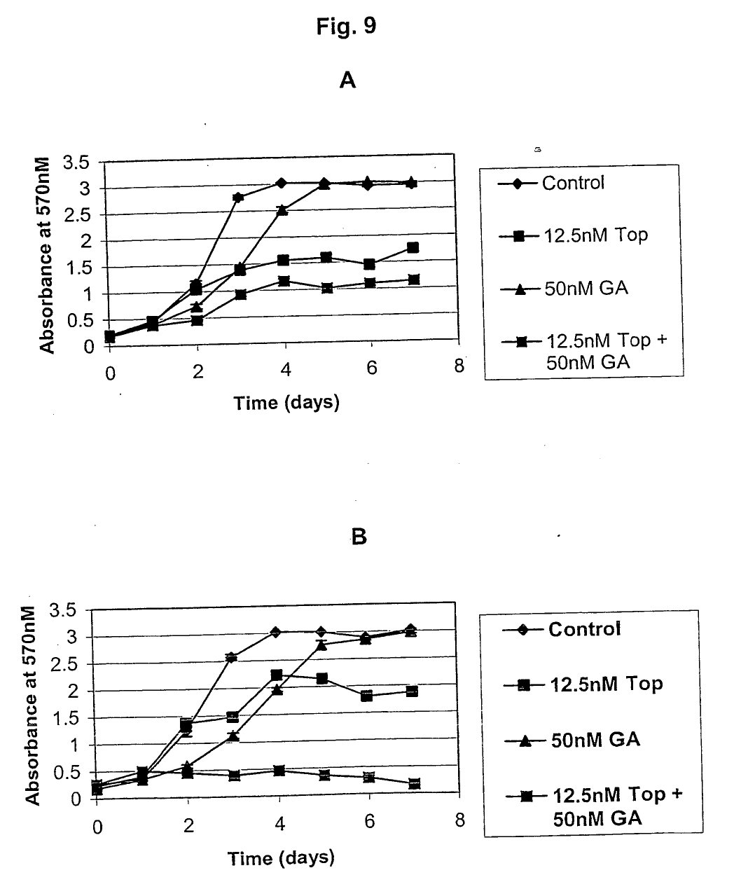

FIG. 9 illustrates growth inhibition caused by topotecan/geldanamycin combination treatment on (A) HCT116 p53 WT cells and (B) p53 KO cells as described in Example 2;

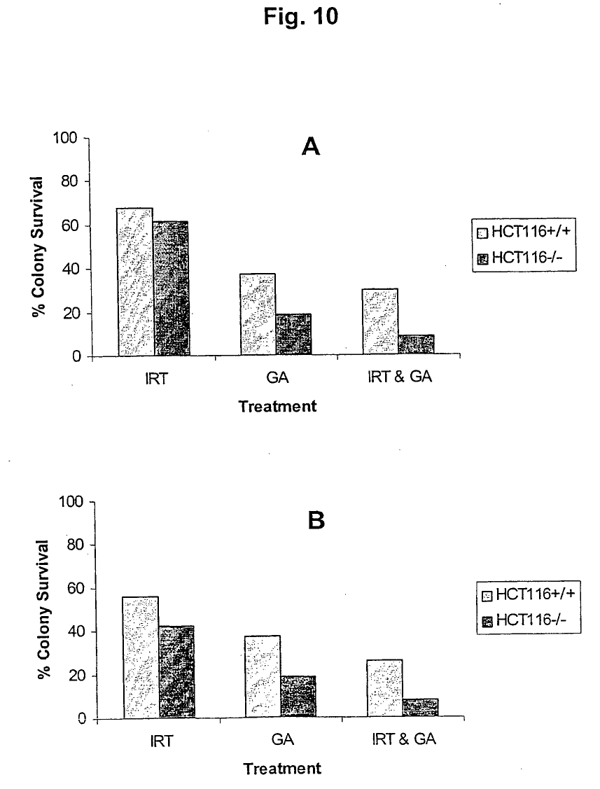

FIGS. 10 illustrates the results of a clonogenic assays investigating the inhibitory effects of Irinotecan (IRT), Geldanamycin (GA) and the combination of IRT plus GA on the growth of both, HCT116 WT and HCT16 KO cells (the effect being independent of p53 status): (A) illustrates combination therapy results obtained with 15μM IRT plus 1.25 μM GA; and (B) shows results for 50 μM IRT plus 1.25 μM GA;

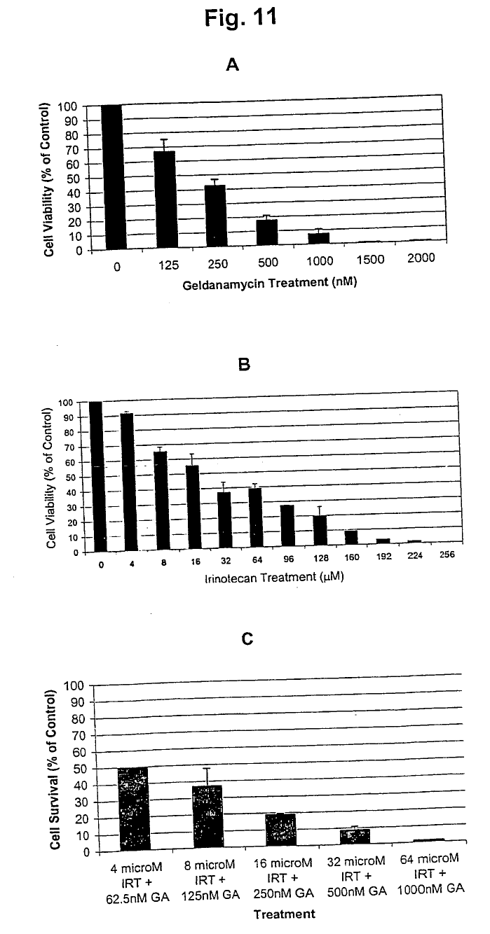

FIGS. 11 illustrates the cell killing response of of HCT116 p53 KO cells after treatment with (A) geldanamycin, (B) irinotecan and (C) geldanamycin/irinotecan in combination, respectively as described in Example 2;

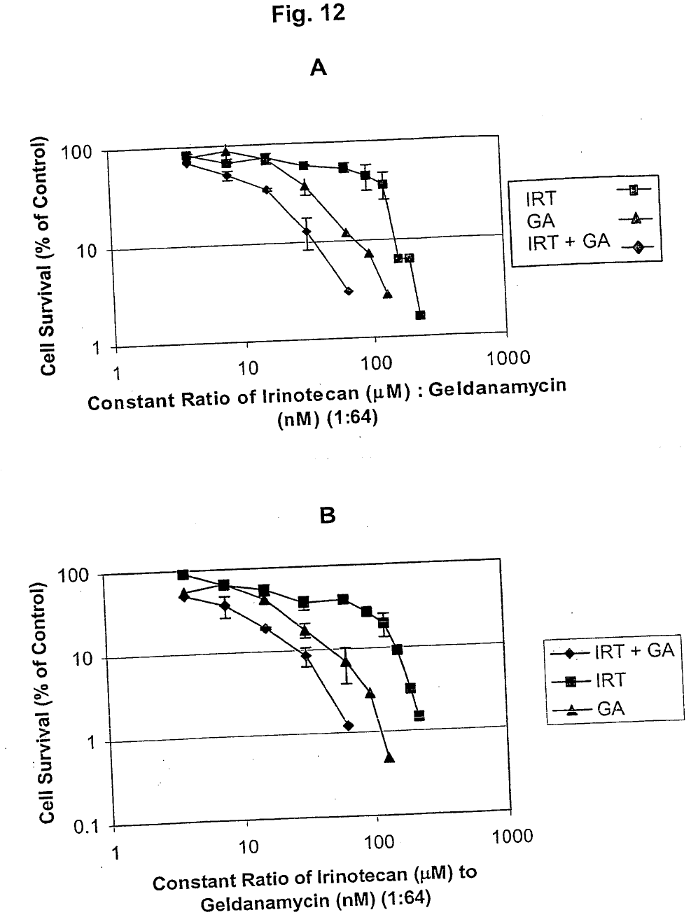

FIG. 12 shows an isobologram illustrating the cell killing response after treatment with irinotecan, geldanamycin and irinotecan/geldanamycin in combination on (A) HCT116 p53 WT cells and (B) p53 KO cells as described in Example 2; and

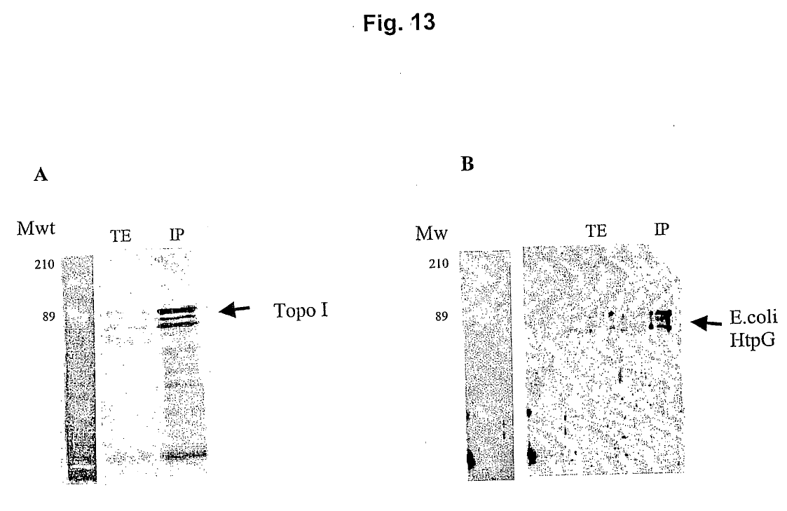

FIGS. 13 illustrates an interaction between topoisomerase I and HtpG as demonstrated by the results of co-immunoprecipitation (IP) western blots of IPs from E.coli extracts wherein: TE=total cell extract, IP=protein complex following immunoprecipitation; (A) shows results obtained from immunoprecipitation with an anti-topoisomerase I antibody in which the western blot was probed with an anti-body against Hsp90 (which recognises the bacterial equivalent HtpG); and (B) shows results obtained from immunoprecipitation with an anti Hsp90 antibody which recognises the bacterial equivalent HtpG whilst the western blot was probed with a topoisomerase I antibody.

### EXAMPLE 1

Experiments were conducted that established HSP90 and Topoisomerase I interact and influence cell growth. This discovery lead the inventor to develop the various aspects of the invention described herein.

1.1 METHODS

Established Cell Culture

The isogenic p53 human colon cancer cell line (WT and KO), HCT116 (see Hwang et al. Nat Med 2001 November 7(11):1255). Cells were maintained in McCoys 5A medium (Sigma) supplemented with 10% foetal calf serum (Gibco) at 37° C. in a 5% CO₂ enriched humidified environment, Penicillin and Streptomycin.

Standard cell lines as above except:

K562 RMPI 1640 (Sigma), SK-MEL-3 McCoys (Sigma), OAW42 DMEM supplemented with 1 mM sodium pyruvate 10 μg/ml insulin & NCI-H125 RPMI 1640 (Sigma), HT29 DMEM (Sigma).

Immunoprecipitations

100 mm dishes were seeded with 3—10⁶ cells and allowed to adhere overnight. Media was placed with fresh media alone (control) or containing a Topo I inhibitor (e.g. Irinotecan) for 24 hours. Cells were washed twice with wash buffer (0.4 mM EDTA, 10 mM sodium fluoride, 10 mM sodium pyrophosphate, 0.4 mM sodium orthovanadate) and incubated on ice with 250 μl cell lysis buffer (50 mM Tris HCl pH 8.0, 425 mM NaCl, 1 mM EDTA, 10 mM sodium fluoride, 0.5 mM sodium orthovanadate, 1% v/v igepal CA-630, 5% w/v deoxycholic acid, 0.1% w/v SDS) containing protease inhibitor cocktail III (Calbiochem). Cells were scraped on ice, sonicated for 30 seconds and cell debris removed by centrifugation at 14,000×g for 30 minutes at 4° C. Cell lysates were then pre-cleared by incubation with 25 μl of 10% w/v protein A sepharose CL-4B (Amersham Pharmacia Biotech) in PBS for 1 hour rotating at 4° C. Samples were spun briefly at maximum speed in a 4° C. benchtop centrifuge and supernatants removed to fresh microfuge tubes. 5 μg of either anti-topoisomerase I or anti-heat shock protein 90β (Labvision) antibodies were added to cell lysates and incubated at 4° C. overnight. 50 μl of 10% w/v protein A sepharose in PBS was added and samples allowed to precipitate by rotating at 4° C. for 1 hour.

Samples were spun briefly at maximum speed in a 4° C. benchtop centrifuge and supernatants discarded. Immunoprecipitates were washed with 250 μl cell lysis buffer, resuspended in 60 μl IPG buffer (7 M urea, 2 M thiourea, 4% w/v CHAPS, 40 mM Tris base, 1% w/v DTT) and analysed by one-dimensional (1-D) electrophoresis.

1-D Electrophoresis and Immunoblotting

Total protein extracts and immunoprecipitations were separated by 7.5% or 12% SDS-PAGE under reducing conditions. Gels were then either stained using Colloidal blue concentrate (Sigma) in 20% v/v methanol or blotted onto nitrocellulose membrane. Blots were probed with either rabbit primary antibodies against human Topoisomerase I or Heat Shock Protein 90β, or mouse primary antibodies against human heat shock protein 70 (Labvision). Anti-rabbit and anti-mouse IgG secondary antibodies conjugated with horseradish peroxidase (DAKO) were detected by Supersignal West Dura Extended Substrate (Pierce) and imaged using a Fluor-S bioimager (BioRad).

1.2 Results

Protein-Protein Interactions

Protein association studies were conducted using 1 dimensional SDS-PAGE analyses of co-immunoprecipitated proteins. Irumunoprecipitation were undertaken with commercial antibodies against the native protein, and binding partners were identified by 1 dimensional SDS-PAGE (see FIG. 1).

The counter precipitation was performed (IP with antibodies against HSP90β) and topoisomerase I was demonstrated to be associated with HSP90. FIG. 2 shows western blots of counter immunoprecipitations and probing of the blots, demonstrating that the corresponding proteins come down in pull down experiments.

Interaction between topoisomerase I and Hsp90 was demonstrated by a further set of immunoprecipitation assays. FIG. 3 A illustrates the results of immunoprecipitation with an anti-topoisomerase I antibody followed by probing of the western blot with an antibody against Hsp90. In addition, this figure shows the outcome of the negative control in which the blot was also probed with an antibody against PKC a, a non-reactive antibody (non-specific binding control). FIG. 3B shows the result of immunoprecipitation with an anti Hsp90 antibody which was followed by probing of the western blot with a topoisomerase I antibody. Overall, this study demonstrates an interaction between topoisomerase I and Hsp90.

Drug Target

The inventor realised that the interaction between these two proteins represents a new drug target and went on to assess the effect of modulators of these proteins in combination for chemotherapy (see Example 2)

### EXAMPLE 2

Example 1 illustrates there was a physical interaction between Topo I and HSP90. The inventor therefore tested the effect of combining drugs that had a specific effect on Topo I and a specific effect on HSP90. The combination of an HSP90 inhibitor and a topoisomerase I inhibitor show a synergistic effect (see below).

2.1 Methods

2.1.1 Growth Inhibition Assay

96 well flat-bottomed plates were seeded with 3×10³ cells per well and allowed to adhere overnight. Media was then replaced with fresh media alone (control) or containing test drugs e.g. 0-100 μM irinotecan, 50 to 200 nM geldanamycin (GA) and combinations of both. At fixed time points, cells were fixed with 3:1 methanol:acetic acid and stained with 0.4% w/v sulforhodamine B (Sigma) in 1% v/v acetic acid for 30 minutes. Plates were then washed twice with 1% v/v acetic acid, the dye solubilised with 100 μl per well of 10 mM Tris pH 10.4 and read at A570nm using a Benchmark microplate reader (BioRad).

2.1.2 Clonogenic Assay

Cells were plated at a density of 1000 cells per well in 6 well plates and allowed to adhere overnight. Cells were treated with e.g. 0.5 to 50 μM VP16, 50 to 1500 nM GA or combinations of the two for 1 hour. Cells were then washed twice with PBS and re-incubated with fresh medium for 10 days. Media was then removed and cells were fixed with 70% v/v methanol for 1 minute. Cells were then stained with 0.2% w/v crystal violet in 70% v/v ethanol for 10 seconds, washed with dH₂O and allowed to air dry. The number of colonies formed of >50 cells each were counted.

Drugs were used in the following concentrations for Growth inhibition and Clonogenic assays:

| | |
|---|---|
| Geldanamycin | 1-1500 nM |
| Irinotecan | 0.01-100 μM |
| Radicicol | 25-350 nM |
| Topotecan | 12.5-800 nM |

2.1.3 Flow Cytometry Protocol for Cell Cycle Analysis.
1. Seed cells eg HCT116+/+ or K562 cells in small petri dish or 6 well plate using 5 ml of 1×10⁶ cells/ml in appropriate medium. For HCT116+/+ cell line use McCoys 5A Medium supplemented with 10% Foetal Calf Serum (FCS) and Penicillin and Streptomycin. For K562 cell line use RPMI 1640 Medium supplemented with 10% FCS and Penicillin and Streptomycin.
2. Leave to attach overnight for adherent cell lines in incubator at 37° C. 5% CO₂ atmosphere.*
   * For suspension cell lines, spin cells down and resuspend at between 2-4×10⁵ cells/ml in medium supplemented with the drug treatment required.
3. Dose with 5 ml of drug/control for required time course in incubator at 37° C. 5% CO₂ atmosphere. 125 nM Geldanamycin, 0.5 μM Irinotecan, or 125 nM Geldanamycin and 0.5 μM Irinotecan combination.
4. After treatment, remove medium from well to a universal tube.**
   ** For suspension cell lines ignore steps 6 and 7.
5. Wash well with 500 μl PBS and remove to same universal.
6. Add 500 μl trypsin and wait for detachment.
7. Add trypsin and cells to universal and rinse out the well with some of the medium from the universal.
8. Spin cells at 4° C. at 2500 rpm for 5 mins.
9. Remove supernatant and resuspend pellet in 500 μl PBS
10. Transfer to Falcon tube and spin at 4° C. at 2500 rpm for 5 minutes
11. Remove supernatant and add 500 μl ice-cold 70% ethanol, and leave in fridge for 2-5 minutes.
12. Spin cells at 4° C. at 2500 rpm for 5 mins.
13. Wash twice in 1 ml PBS.
14. Add 40 μl of 100 μg/ml ribonuclease A for 5 mins at room temperature.
15. Add 400 μl of 50 μg/ml propidium iodide (Sigma) and incubate for 15 minutes.
16. Analyse on FACSVantage SE (Becton Dickinson) using 488 nm laser for excitation, and collecting fluorescence above 585 nm (FL-2). Collect data using CellQuest Pro v4.0. Analyse data using Mod Fit LT v3.0
   2.1.4 Isobolar Relations

The isobolar relations were calculated to quantify the synergistic combination of the two agents used according to the invention.

The isobolar relation is calculated in the light of the fact that two drugs used in combination may produce enhanced or reduced effects. The degree of enhancement or reduction is measured from the interaction index (γ), defined by the isobolar relation, which indicates the changed potency of the combination.
(a/A)+(b/B)=γ
Where:A=drug A alone; B=drug B alone; and a, b=combination dose to produce desired effect

If γ 1 =additive; <1=super-additive (synergistic); and >1=sub-additive

2.2 Results

2.2.1 Growth Inhibition Assay

Example 1 illustrates that the interaction between these two proteins represents a new drug target. The effectiveness of modulating the two proteins in chemotherapy was tested using inhibitors of HSP 90 and inhibitors of Topo I in combination.

The beneficial effects of inhibitors of HSP 90 and inhibitors of Topo I are shown in FIGS. 4-9.

The inhibitory effects of Irinotecan alone or Geldanamycin alone on cell growth are shown in FIG. 4 and FIG. 5, respectively. Moreover, the synergistic inhibitory effect of the combination of Irinotecan and Geldanamycin on cell proliferation is shown in FIGS. 6 and 7.

Synergistic effects were also seen when Radicicol was combined with Irinotecan (see FIG. 8) and when Topotecan was combined with Geldanamycin (see FIG. 9).

2.2.2 Clonogenic Assay

In the clonogenic assays, cell killing, showed a similar synergistic effect when a combination of HSP 90 and Topo I inhibitors was used. The combination of Irinotecan and Geldanamycin resulted in less colony survival than treatment with single drugs (FIG. 10).

In FIG. 11, the cell killing response of HCT116 p53 cells after treatment with two agents is shown. Overall, this produced a synergistic effect and resulted in cell killing at concentrations, where there is little or no effect with single drugs. Thus, cell killing was found to be at least 3-5 times greater fro combination therapy than for the drug used in isolation. The action was also found to be independent of p53 status.

Flow cytometry experiments indicate that greater DNA damage may be seen with combination therapy according to the invention.

2.2.3 Isobolar Relations

FIG. 12 shows an isobologram illustrating the cell killing response after treatment with irinotecan, geldanamycin and irinotecan/geldanamycin in combination on (A) HCT116 p53 WT cells and (B) p53 KO cells as described in Example 2; and

It will therefore be appreciated that the combination of the two agents produces a synergistic effect (i.e. cell killing at concentrations, where there is little or no effect with single drugs)

### EXAMPLE 3

Experiments were conducted to illustrate the efficacy of the combination therapy according to the present invention for destroying mircroorganisms

3.1 Methods

An E.coli extract was generated from a 50 ml over night culture of bacteria (shaking culture at 37° C.). The culture was harvested and resuspended in lysis buffer 0.5 ml and sonicated to disrupt the cells, the extract was then cleared of debris by centrifugation at 12,000 g for 15 minutes the cleared extract was then used for the immunoprecipitation (all these procedures were carried out at 4° C.).

Co-immunoprecipitation (IP) western blots of IPs from E.coli extracts were then carried out. Initially, immunoprecipitation with an anti-topoisomerase I antibody the western blot was probed with an anti-body against Hsp90 (which recognises the bacterial equivalent HtpG). This was accompanied by an IP with anti Hsp90 antibody (which recognises the bacterial equivalent HtpG) whose western blot was probed with a topoisomerase I antibody.

3.2 Results

This example demonstrates that HSP90 and Topoisomerase I interact and influence microbial cell growth and susceptibility to chemotherapy.

FIG. 13 shows western blots of immunoprecipitation and counter immunoprecipitation probed with the complementary antibodies, demonstrating that the corresponding proteins come down in pull down experiments and thereby illustrating that Hsp90 and Topoisomerase I homologues in bacteria interact and influence cell growth and death.

1. A method of administering chemotherapy, comprising:
administering a first agent that attenuates Topoisomerase I (Topo I) activity; and
administering a second agent that inhibits Heat Shock Protein 90 (HSP90) activity,
wherein the first agent and the second agent are administered either contemporaneously or sequentially.

2. The method of claim 1 wherein the first agent is a compound selected from the group consisting of:
(i) compounds that bind to Topo I and inhibit its activity,
(ii) compounds which prevent the transcription, translation or expression of Topo I,
(iii) compounds which inhibit release of Topo I from intracellular stores, and
(iv) compounds which increase the rate of degradation of Topo I.

3. The method of claim 1 wherein the first agent is a cleavable-complex inhibitor.

4. The method of claim 1 wherein the first agent is Camptothecin.

5. The method of claim 1 wherein the first agent is Topotecan.

6. The method of claim 1 wherein the first agent is Irinotecan.

7. The method of claim 1 wherein the first agent is Camptostar (CPT-11).

8. The method of claim 1 wherein the first agent is Gemcitabine.

9. The method of claim 1 wherein the second agent is a compound selected from the group consisting of:
(i) compounds that bind to HSP 90 and inhibit its activity,
(ii) compounds which prevent the transcription, translation or expression of HSP 90,
(iii) compounds which inhibit release of HSP 90 from intracellular stores, and
(iv) compounds which increase the rate of degradation of HSP 90.

10. The method of claim 9 wherein the second agent is Geldanamycin.

11. The method of claim 10 wherein the second agent is 17-Allylamino, 17-demethoxygeldanamycin (17AAG) or CNF-101.

12. The method of claim 9 wherein the second agent is Radicicol.

13. The method of claim 1 wherein the chemotherapy is for cancer treatment.

14. The method of claim 13 for the treatment of solid tumours.

15. The method of claim 14 for the treatment of bowel cancer, small cell and non-small cell lung cancer, head and neck cancer, breast cancer, bladder cancer or malignant melanoma.

16. The method of claim 15 for the treatment of paediatric tumours.

17. The method of claim 13 for the treatment of neuroblastoma, leukaemias and/or lymphomas.

18. The method of claim 1 wherein the chemotherapy is for:
antibacterial treatments,
antifungal treatments,
antiparasitic treatments,
the treatment of AIDS/HIV,
the treatment of multiple sclerosis, or
the killing and inhibition of proliferation of any organism.

19. The method of claim 1 wherein the chemotherapy is for prophylactic treatment.
